(19) United States
(12) Patent Application Publication
(10) Pub. No.: US 2026/0079978 A1
(43) Pub. Date: Mar. 19, 2026

(54) MEDICAL RARE EVENT PREDICTION METHOD, APPARATUS, AND STORAGE MEDIUM FOR MULTIMODAL DATA

(71) Applicant: WEST CHINA HOSPITAL, SICHUAN UNIVERSITY, Chengdu City (CN)

(72) Inventors: JING TAN, Chengdu City (CN); XIN SUN, Chengdu City (CN); DONGLIN XIE, Chengdu City (CN); CHUNRONG LIU, Chengdu City (CN); YIQUAN XIONG, Chengdu City (CN); YAN REN, Chengdu City (CN)

(21) Appl. No.: 18/900,464

(22) Filed: Sep. 27, 2024

(30) Foreign Application Priority Data
Sep. 14, 2024 (CN) 2023114989757

(51) Int. Cl. G06F 16/33; G06F 40/284; G16H 10/60

(52) U.S. Cl. CPC G06F 16/3344; G06F 40/284; G16H 10/60

(57) ABSTRACT

A medical rare event prediction method, apparatus and storage medium of multimodal data includes: on the basis of the existing model of the transformer, by preprocessing the data, the absence of imputed data, semantic feature extraction on structured data simultaneously leveraging powerful modeling capabilities exhibited by the model of transformers in multiple domains such as natural language processing and computer vision, again by extracting unstructured data, that is, the semantic characteristics of the text data, the semantic characteristics of the structured data and the unstructured data are fused, the prediction of the medical rare event is performed based on the fused characteristics, and the prediction of the medical rare event of the multimodal data is achieved using the structured data and the unstructured data, and the prediction accuracy is improved.

### TECHNICAL FIELD

The present disclosure relates to the field of medical rare event prediction, and in particular to a medical rare event prediction method, apparatus and storage medium of multimodal data.

### BACKGROUND

The medical rare event prediction task is essentially a binary classification task, i.e. learning the data feature distribution with a machine learning or deep learning model, and predicting whether a medical rare event will occur, and the data in the medical actual scenario presents a multimodal form, including structured tabular data as well as unstructured textual data, and thus modeling based on the multimodal data is required to predict the medical rare event.

Current medical rare event prediction models are mostly modelled based on structured data of a single modality, it is mainly based on machine learning models, such as Random Forest, xgboost, lightgbm, and the like. Machine learning models based on tree structures have good interpretability, as a decision path from the root node to the leaf node of the tree, and a good interpretation of the decision basis at the time of prediction by the model.

With the development of deep learning in recent years, with stronger fitting capability based on deep learning, neural network models such as multilayer perceptron, deepfm, etc. are also commonly used for structured data modeling, such as foreign teams propose methods for predicting medical rare events using deep convolutional neural networks vgg-16 for modeling, and recently, transformer-based models have demonstrated strong modeling capabilities in the fields of image and natural language processing, etc.

But there is still less work to apply a transformer to structured data, the method of modeling structured data using a transformer, but is not able to handle missing values in structured data, deep learning, while having a stronger fitting capability, is weaker in model interpretively than machine learning models, and is not able to utilize data from other modalities, such as unstructured textual data.

### SUMMARY

In view of the above, a medical rare event prediction method, apparatus and storage medium of multimodal data is provided.

A medical rare event prediction method of multimodal data, including:
textual data and structured data related to patients from electronic medical records are acquired, the structured data including structured category feature data and structured numerical feature data;
structured category feature data and structured numeric feature data are pre-processed, and textual data for filling missing values in the structured category feature data and structured numeric feature data;
pre-processed structured category feature data in Embedding module is projected to obtain vectorized structured category feature data;
pre-processed structured numerical feature data is combined to obtain a combined matrix, inputting the combined matrix into a preset linear layer for dimensionalization to obtain vectorized structured numerical feature data;
a mask matrix with structured numerical feature data based on the structured category feature data is initialized, taking values of the initialized mask matrix with missing values imputed in structured numeric feature data according to the structured category feature data to obtain a target mask matrix;
vectorized structured category feature data is concatenated and the vectorized structured numerical feature data to obtain structured features, inputting the structured features into a pre-built transformer model, performing feature construction on the structured features through a multi-head self-attention mechanism of the pre-built transformer model, calculating weight scores by filtering out the influence of missing features through the target mask matrix, performing weighted fusion of the projected structured features according to the weight scores, and obtaining output features of the pre-built transformer model;
the output features of the pre-built transformer model is taken as inputs to a gated mlp module and taking the output features of the gated mlp module as global semantic features of the structured data;
textual data into a pre-trained language model is inputted, encoding the textual data by the pre-trained language model, taking features output by the pre-trained language model as semantic features of the textual data;
global semantic features of the structured data and semantic features of textual data resulting in fused features are concatenated, the fused features is inputted into a linear layer through which probabilities of user medical rare events are output.

Further, structured category feature data is pre-processed, the structured numeric feature data, and the textual data to fill missing values in the structured category feature data and the structured numeric feature data includes:
character type of each category feature in the structured category feature data is converted to an integer type, each category being represented by a different integer;
when a missing value occurs for any one category feature, populating with the category total for that category feature;
the median of each numerical feature in the structured numerical feature data is populated when missing values occur;
a mean-standard deviation normalization process is performed on the populated structured numerical feature data;
numerical features in the textual data is extracted by way of regular expression matching, adding the extracted numerical features to the structured numerical feature data.

Further, pre-processed structured category feature data is projected in an Embedding module resulting in vectorized structured category feature data includes:
an offset is added to each class feature in the pre-processed structured class feature data, the offset being equal to a total number of classes of own pre-class features;
values of a shifted class features is taken as indices to retrieve corresponding row vectors in the Embedding matrix of the Embedding module, projecting all of the shifted class features to a matrix of a target dimension, and taking a matrix of the target dimension as vectorized structured class feature data.

Further, feature construction of the structured features by a multi-head self-attention mechanism of the pre-built transformer model, calculating weight scores by filtering out the influence of missing features through the target mask matrix, performing weighted fusion of the projected structured features according to a weight scores, and obtaining an output features of the pre-built transformer model includes:
the multi-head self-attention mechanism of the transformer model initializes three linear layers, the three linear layers including a parameter matrix Wq, a parameter matrix Wk, and a parameter matrix Wv, respectively, the three linear layers further including biases bq, bk, and bv, respectively;
three linear layers of the structured features are projected by their own parameter matrices and biases, resulting in a query vector, a key vector, and a value vector, respectively;
and a weighted fusion is performed by the query vector, key vector, value vector, and weight scores to obtain the output features of the pre-built transformer model.

Further, textual data is inputted into a pre-trained language model, encoding the textual data by the pre-trained language model, taking features output by the pre-trained language model as semantic features of the textual data includes:
word tokenization on the textual data is performed to obtain a word tokenization sequence, and adding special word tokenization [CLS] and [SEP] at the beginning and end of the word tokenization sequence, respectively;
each token is converted into a corresponding token id according to a lexicon of the pre-trained language model, indexing a word vector of each token from an embedding matrix of the pre-trained language model with the token id;
each word vector plus is inputted a corresponding position coding into the pre-trained language model, extracting features of textual data by a self-attention module, and taking word vectors F_text output by [CLS] word tokenization as semantic features of an entire piece of textual data.

Further, a mask matrix with structured numeric feature data based on the structured category feature data is initialized, taking the initializing mask matrix with missing values imputed in the structured numeric feature data according to the structured category feature data to obtain a target mask matrix includes:
if n class features are included in the structured class feature data, m numerical features are included in the structured numerical feature data, the initialization mask matrix has a size of (n+m)×(n+m), each element in the initialized mask matrix takes a value of 0 or 1 on a condition that: M (i, j) represents whether a semantic relatedness between an i-th feature and a j-th feature needs to be calculated, when the j-th feature is missing, then let M (i, j)=0, otherwise M (i, j)=1, taking all elements of the initialized mask matrix after obtaining a target mask matrix.

Further, a medical rare event prediction apparatus is provided, the apparatus including:
a data acquisition module to acquire textual data and structured data related to a patient from electronic medical records, the structured data including structured category feature data and structured numerical feature data;
a preprocessing module for preprocessing the structured category feature data, structured numeric feature data and textual data for filling missing values in the structured category feature data and structured numeric feature data;
a category vectorization module: configured to project the pre-processed structured category feature data in the Embedding module, resulting in vectorized structured category feature data;
a numerical vectorization module: configured to combine the pre-processed structured numerical feature data to obtain a combined matrix, and input the combined matrix into a preset linear layer for dimensionalization to obtain vectorized structured numerical feature data;
a filtering module: configured to initialize a mask matrix with a structured numeric feature data based on the structured category feature data, take values on the initialized mask matrix with missing values padded in the structured numeric feature data according to the structured category feature data to obtain a target mask matrix;
a weighted fusion module: for concatenating the vectorized structured category feature data and the vectorized structured numerical feature data to obtain structured features, inputting the structured features into a pre-built transformer model, performing feature construction on the structured features through a multi-head self-attention mechanism of the pre-built transformer model, calculating weight scores by filtering out the influence of missing features through the target mask matrix, performing weighted fusion of the projected structured features according to the weight scores, and obtaining output features of the pre-built transformer model;
a structured data semantic acquisition module configured to take the output features of the pre-built transformer model as input to the gated mlp module and the output features of the gated mlp module as global semantic features of the structured data;
a textual data semantic acquisition module: configured to input the textual data into a pre-trained language model, encode the textual data by the pre-trained language model, take features output by the pre-trained language model as semantic features of the textual data;
a prediction module for concatenating global semantic features of the structured data and semantic features of textual data resulting in fused features, inputting the fused features into a linear layer through which probabilities of user medical rare events are output.

Further, a storage medium is provided, the storage medium storing a computer program, when executed by a master, implements various steps in a medical rare event prediction method of multimodal data according to any one of the above method.

By preprocessing the data, the absence of imputed data, semantic feature extraction on structured data simultaneously leveraging powerful modeling capabilities exhibited by the model of transformer in multiple domains such as natural language processing and computer vision, again by extracting unstructured data, that is, the semantic characteristics of the textual data, the semantic characteristics of the structured data and the unstructured data are fused, the prediction of the medical rare event is performed based on the fused characteristics, and the prediction of the medical rare event of the multimodal data is achieved using the structured data and the unstructured data, and the prediction accuracy is improved.

### BRIEF DESCRIPTION OF THE DRAWINGS

FIG. 1 is a flow diagram illustrating a medical rare event prediction method of multimodal data according to some embodiments.

FIG. 2 is a schematic diagram illustrating a transformer-based structured modeling according to some embodiments.

FIG. 3 is a schematic diagram illustrating semantic extraction of unstructured data according to some embodiments.

FIG. 4 is a system schematic diagram illustrating a medical rare event prediction apparatus of multimodal data according to some embodiments.

### DETAILED DESCRIPTION OF THE EMBODIMENTS

Reference will now be made in detail to exemplary embodiments, examples of which are illustrated in the accompanying drawings. Where the following description refers to the drawings, unless otherwise indicated, the same numbers in different drawings refer to the same or similar elements. The embodiments described in the following exemplary embodiments do not represent all embodiments consistent with the present disclosure. Rather, they are merely examples of apparatus and methods consistent with some aspects as detailed in the appended claims.

### Embodiment One

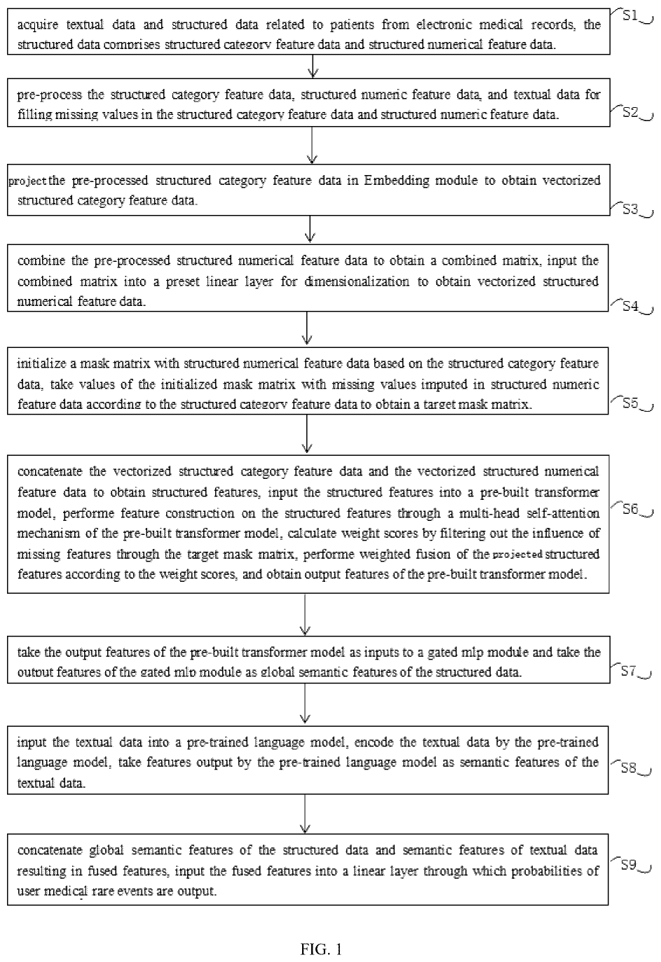

FIG. 1 is a flow diagram illustrating a medical rare event prediction method based on multimodal data according to an exemplary embodiment, the method including:
in step S1, textual data and structured data related to patients from electronic medical records are acquired, the structured data including structured category feature data and structured numerical feature data;
in step S2, structured category feature data and structured numeric feature data are pre-processed, and textual data for filling missing values in the structured category feature data and structured numeric feature data;
in step S3, pre-processed structured category feature data in Embedding module is projected to obtain vectorized structured category feature data;
in step S4, pre-processed structured numerical feature data is combined to obtain a combined matrix, inputting the combined matrix into a preset linear layer for dimensionalization to obtain vectorized structured numerical feature data;
in step S5, a mask matrix with structured numerical feature data based on the structured category feature data is initialized, taking values of the initialized mask matrix with missing values imputed in structured numeric feature data according to the structured category feature data to obtain a target mask matrix;
in step S6, vectorized structured category feature data is concatenated and the vectorized structured numerical feature data to obtain structured features, inputting the structured features into a pre-built transformer model, performing feature construction on the structured features through a multi-head self-attention mechanism of the pre-built transformer model, calculating weight scores by filtering out the influence of missing features through the target mask matrix, performing weighted fusion of the projected structured features according to the weight scores, and obtaining output features of the pre-built transformer model;
in step S7, the output features of the pre-built transformer model is taken as inputs to a gated mlp module and taking the output features of the gated mlp module as global semantic features of the structured data;
in step S8, textual data into a pre-trained language model is inputted, encoding the textual data by the pre-trained language model, taking features output by the pre-trained language model as semantic features of the textual data;
in step S9, global semantic features of the structured data and semantic features of textual data resulting in fused features are stitched, the fused features is inputted into a linear layer through which probabilities of user medical rare events are output.

As can be appreciated, textual data and structured data related to a patient is acquired from electronic medical records, the structured data including structured category feature data and structured numerical feature data; pre-processing the structured category feature data, structured numeric feature data, and textual data for filling missing values in the structured category feature data and structured numeric feature data; projecting the pre-processed structured category feature data in an Embedding module to obtain vectorized structured category feature data; combining the pre-processed structured numerical feature data to obtain a combination matrix, and inputting the combination matrix into a preset linear layer for dimensionalization to obtain vectorized structured numerical feature data, specifically as follows:
since the numerical features are continuous in value, they cannot be vectorized directly by the Embedding module like the categorical features, let m numerical features be z₁, z₂, . . . , zₘ respectively, after being subjected to the normalization process of the pre-processing step by the following steps:
randomly initializing a linear layer including a weight part W (W is a matrix of size 1×d), and a bias part bias (bias is a randomly initialized floating point number).

Numerical features are up-dimensioned, combining numerical features z₁, z₁, . . . , zₘ into a matrix

$$Z = [ \begin{matrix}{Z}_{1} \\ {Z}_{2} \\ … \\ {Z}_{m}\end{matrix} ]$$

with dimensions of size m×1 and then input into a linear layer, computed as follows:

$$\overline{Z} = ZW + bias$$

wherein Z̅ is a numerical feature matrix after upsizing, and has a size of m×d, and each row corresponds to a vector feature after z₁, z₂, . . . , zₘ upsizing, respectively;
initializing a mask matrix with structured numeric feature data based on the structured category feature data, taking values of the initialized mask matrix with missing values imputed in structured numeric feature data according to the structured category feature data to obtain a target mask matrix; concatenating the vectorized structured category feature data and the vectorized structured numerical feature data to obtain structured features, inputting the structured features into a pre-built transformer model, performing feature construction on the structured features through a multi-head self-attention mechanism of the pre-built transformer model, calculating weight scores by filtering out the influence of missing features through the target mask matrix, performing weighted fusion of the projected structured features according to the weight scores, and obtaining output features of the pre-built transformer model; taking the output features of the pre-built transformer model as inputs to a gated mlp module and taking the output features of the gated mlp module as global semantic features of the structured data; inputting the textual data into a pre-trained language model, encoding the textual data by the pre-trained language model, taking features output by the pre-trained language model as semantic features of the textual data; concatenating global semantic features of the structured data and semantic features of textual data, resulting in fused features, which are input into a linear layer through which probabilities of user medical rare events are output as follows:
the data information provided by a single modality is limited, so joint modeling with data of different modalities is used to predict targets, the global semantics of the structured features and the semantics of the textual features obtained above are concatenated, and then input to the linear layer for target prediction, which is calculated as follows:

$$p = sigmoid ( W [ {F}_{struc} , {F}_{text} ] )$$

wherein p represents a probability predicted by the model, when p is greater than a set threshold, it is determined that a medically rare event will occur for the input sample, otherwise the medically rare event will not occur.

The present disclosure is based on the model of the existing transformer, by preprocessing the data, the absence of imputed data, semantic feature extraction on structured data simultaneously leveraging powerful modeling capabilities exhibited by the model of transformers in multiple domains such as natural language processing and computer vision, again by extracting unstructured data, that is, the semantic characteristics of the textual data, the semantic characteristics of the structured data and the unstructured data are fused, the prediction of the medical rare event is performed based on the fused characteristics, and the prediction of the medical rare event of the multimodal data is achieved using the structured data and the unstructured data, and the prediction accuracy is improved.

The pre-processing the structured category feature data, the structured numeric feature data, and the textual data to fill missing values in the structured category feature data and the structured numeric feature data includes:
converting the character type of each category feature in the structured category feature data to an integer type, each category being represented by a different integer;
when a missing value occurs for any one category feature, populating with the category total for that category feature;
populating by the median of each numerical feature in the structured numerical feature data when missing values occur;
performing a mean-standard deviation normalization process on the populated structured numerical feature data;
extracting numerical features in the textual data by way of regular expression matching, adding the extracted numerical features to the structured numerical feature data;
structured textual preprocessing: converting a character type of a category feature to an integer type, e.g. category feature x a total of c categories, then each category is represented by an integer 0, 1, . . . , c−1, respectively, and for the missing values in the category feature, the total number of categories is populated, e.g. for a category feature x, if x has three categories in total, denoted by 0, 1, 2, respectively, and if its category denoted by 2 is missing, the category feature x is populated with missing values by 3, i.e. the category feature x denotes the category by the integer 3, not denoted by 0, 1 anymore; for missing values of a numerical feature, the median of that feature is used for padding, which is less susceptible to outliers than mean padding.

Normalization process: mean-standard deviation normalization was performed for numerical-type features, let the numerical features be z, and the normalized features were as follows:

$$\overline{z} = \frac{z - mean}{std}$$

where Z̅ represents the numerical feature after normalization, mean represents the mean of z, std represents the standard deviation of z.

Unstructured textual preprocessing: for textual in a medical electronic medical record, among them are some summary textual data of the doctor and some index data of the patient exam, for index data, it is usually presented in decimal form, which for current pre-trained language models is more difficult to model and not easy to feature extraction, so that numerical features in textual data are extracted by way of regular expression matching and complemented with numerical features in structured data.

The projecting the pre-processed structured category feature data in an Embedding module resulting in vectorized structured category feature data includes:
adding an offset to each class feature in the pre-processed structured class feature data, the offset being equal to a total number of classes of own pre-class features;
taking the values of the shifted class features as indices to retrieve corresponding row vectors in the Embedding matrix of the Embedding module, projecting all of the shifted class features to a matrix of a target dimension, and taking the matrix of the target dimension as vectorized structured class feature data.

It can be appreciated that the class features are encoded in a pre-processing process, converting a character type to an integer type, it is then vectorized in the modeling phase, first let n class features be x₁, x₂, . . . , xₙ, there are a₁, a₂, . . . , aₙ categories respectively, after pre-processed encoding, x₁ has a value in the range of 0, 1, . . . a₁−1, x₂ is in the range of 0, 1, . . . , a₂−1, xₙ is in the range 0, 1, . . . , aₙ−1, and the category features are added with an offset so that the values between each category feature do not repeat, and for category xₜ, the offset is equal to the total number of categories of the first t−1 category features, so that the range of values for x₂ plus offset a₁ becomes a₁, a₁+1, . . . , a₁+a₂−1, and the range of values for category feature xₙ plus offset (a₁+a₂+ . . . +aₙ₋₁) is: (a₁+a₂+ . . . +aₙ₋₁), (a₁+a₂+ . . . +aₙ₋₁+1), . . . , (a₁+a₂+ . . . +aₙ₋₁+aₙ−1).

Initializing an Embedding module including a randomly initialized (a₁+a₂+ . . . +aₙ)×d size Embedding matrix, where d is the dimension of the vector.

Taking the value of the category feature as an index to retrieve the corresponding row vector of the Embedding matrix, e.g., 2 for x₁, projecting to the second row vector of the matrix, x₂ for a₁+1, projecting to the a₁+1 row vector of the matrix, and finally obtaining a matrix of dimension n xd after projecting x₁, x₂, . . . , xₙ as the category feature projected feature X̅.

The feature construction of the structured features by a multi-head self-attention mechanism of the pre-built transformer model, calculating weight scores by filtering out the influence of missing features through the target mask matrix, performing weighted fusion of the projected structured features according to the weight scores, and obtaining the output features of the pre-built transformer model includes:
the multi-head self-attention mechanism of the transformer model initializes three linear layers, the three linear layers including a parameter matrix Wq, a parameter matrix Wk, and a parameter matrix Wv, respectively, the three linear layers further including biases bq, bk, and bv, respectively;
the three linear layers project the structured features by their own parameter matrices and biases, resulting in a query vector, a key vector, and a value vector, respectively;
weighted fusion of the query vector, the key vector, the value vector, and the weight scores to obtain output features of the pre-built transformer model.

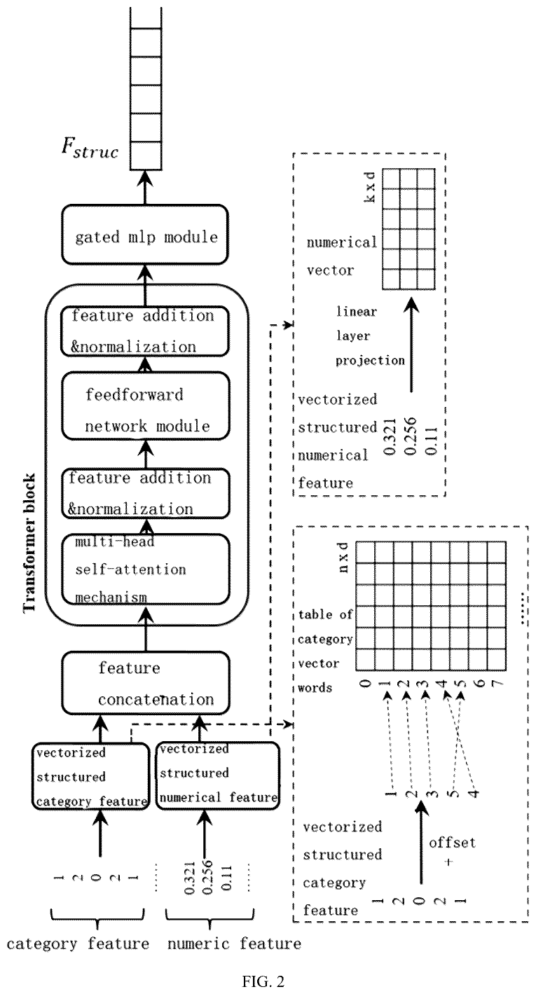

It will be appreciated that, as shown in FIG. 2, vectorized category features and numerical features are first concatenated to obtain structured features

$$F = [ \begin{matrix}\overline{X} \\ \overline{Z}\end{matrix} ] ,$$

Its characteristic dimension is (n+m)×d, F is then taken as the input to the transformer module, feature extraction is performed using a transformer's multi-head self-attention mechanism, the self-attention mechanism essentially exploits semantic relatedness between features for weighted fusion of features, but since there will typically be a lack of features in the structured data, weighted fusion of missing features directly may introduce noise bias, therefore, the missing features are filtered for missing values based on the mask matrix M obtained above, and the weighted scores are obtained. The self attention module first initializes three linear layers to project the query, key, and value vectors, respectively. The three linear layers include the Wq, Wk, Wv parameter matrices and the biases bq, bk, bv parameter matrices, respectively, which are calculated as follows:

$${F}_{q} = {FW}_{q} + {b}_{q}$$

$${F}_{k} = {FW}_{k} + {b}_{k}$$

$${F}_{v} = {FW}_{v} + {b}_{v}$$

$${F}_{output} = softmax ( {F}_{q} {F}_{k}^{T} + \overline{M} ) {F}_{v}^{T}$$

Where M̅ is the weighted score and Fₒᵤₜₚᵤₜ represents the features output by the transformer module.

While Fₒᵤₜₚᵤₜ will be the input to the gated mlp module, gated mlp is a plug-and-play parallel module proposed by Google in 2021, global/local spatial interaction is possible, and has linear complexity, inspired in a multi-axis self-attention module, efficient global/local information interaction on low resolution feature projects is possible, the present disclosure takes the output feature F_{struc} of the gated mlp network as a global semantic feature of the structured data when multiple picture generation tasks reach SOTA, can be used on high resolution underlying visual tasks, and at the same time have “fully convolutional” properties without sacrificing important properties of the global receptive field.

The inputting the textual data into a pre-trained language model, encoding the textual data by the pre-trained language model, taking features output by the pre-trained language model as semantic features of the textual data includes:
performing word tokenization on the textual data to obtain a word tokenization sequence, and adding special word tokenization [CLS] and [SEP] at the beginning and end of the word tokenization sequence, respectively;
converting each token into a corresponding token id according to a lexicon of the pre-trained language model, indexing a word vector of each token from an embedding matrix of the pre-trained language model with the token id;
inputting each word vector after adding a corresponding position coding into the pre-trained language model, extracting features of textual data by a self-attention module, and taking word vectors F_text output by [CLS] word tokenization as semantic features of an entire piece of textual data.

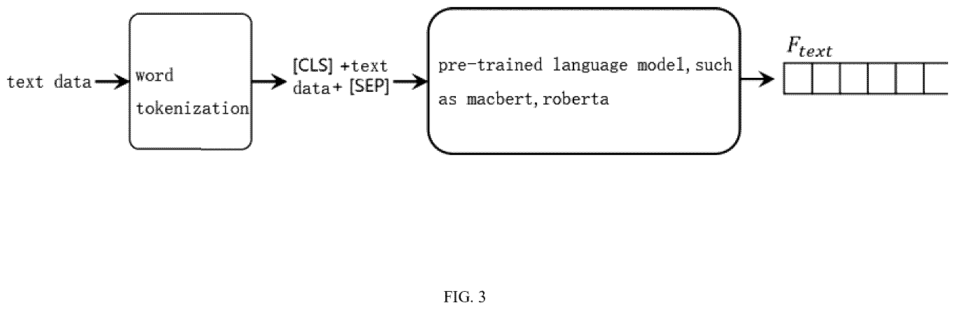

It can be appreciated that as shown in FIG. 3, textual data and structured data belong to different modalities, and the textual is in natural language, cannot be directly processed by a computer, we therefore use a pre-trained language model to encode textual data to extract features, first word tokenization of the input textual results in a word tokenization sequence, add special participates [CLS] and [SEP] beginning-to-end, respectively, for example, doing word tokenization for the textual “I'm fishing at the river” results in [“I”, “am”, “fishing”, “at”, “the”, “river”], then adding the special token results in the sequence [“[CLS]”, “I”, “am”, “fishing”, “at”, “the”, “river”, “[SEP]”], then convert each token into a corresponding id according to a lexicon of a pre-trained model, and index the corresponding word vector from the embedding matrix of the pre-trained model with id, then input the word vector into the pre-trained language model after adding position coding, extract features of the textual data with its transform module, and take the word vector F text output from [CLS] tokenization as semantic characterization of the whole piece of text.

The initializing a mask matrix with structured numeric feature data based on the structured category feature data, taking the initializing mask matrix with missing values imputed in the structured numeric feature data according to the structured category feature data to obtain a target mask matrix includes:
if n class features are included in the structured class feature data, m numerical features are included in the structured numerical feature data, the initialization mask matrix has a size of (n+m)×(n+m), each element in the initialized mask matrix takes a value of 0 or 1, taking a condition that: M (i, j) represents whether a semantic relatedness between an i-th feature and a j-th feature needs to be calculated, when the j-th feature is missing, then let M (i, j)=0, otherwise M (i, j)=1, taking all elements of the initialized mask matrix after obtaining a target mask matrix.

It can be appreciated that the category-based features are x₁, x₂, . . . , xₙ, numerical features z₁, z₂, . . . , zₘ, initialize a matrix M of size mask (n+M)×(n+M), where each element takes the value 0 or 1, M (i, j) denotes whether the semantic relatedness between the ith feature and the jth feature needs to be computed, when the jth feature is missing, then let M (i, j)=0, otherwise M (i, j)=1, e.g. sample s a total of three numerical features x1, x2, x3, three categorical features z1, z2, z3, missing on feature x2, z3, then the corresponding mask matrix is as follows:

$$M = [ \begin{matrix}1 & 0 & 1 & 1 & 1 & 0 \\ 1 & 0 & 1 & 1 & 1 & 0 \\ 1 & 0 & 1 & 1 & 1 & 0 \\ 1 & 0 & 1 & 1 & 1 & 0 \\ 1 & 0 & 1 & 1 & 1 & 0 \\ 1 & 0 & 1 & 1 & 1 & 0\end{matrix} ]$$

It is worth noting that the missing value filtering of missing features by the target mask matrix described in the above results in weighted scores as follows:

$$\overline{M} = - 1 {e}^{9} ( 1 - M )$$

### Embodiment Two

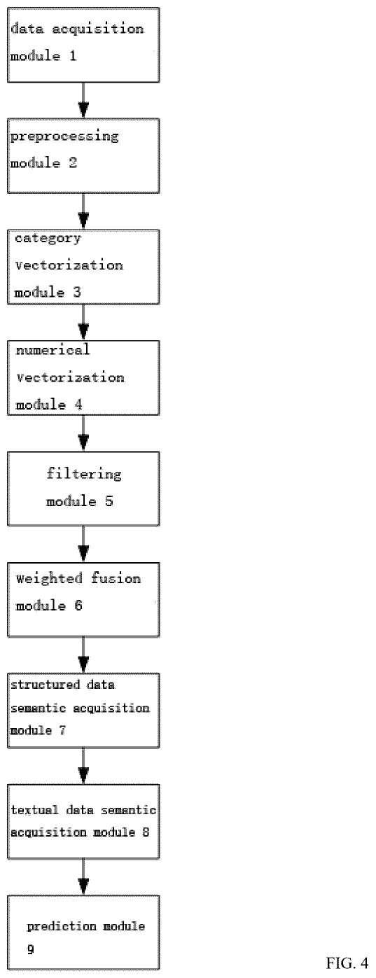

FIG. 4 is a system diagram illustrating a medical rare event prediction apparatus based on multimodal data according to another exemplary embodiment, the apparatus including:
data acquisition module 1: for acquiring textual data and structured data related to a patient from an electronic medical record, the structured data including structured category feature data as well as structured numerical feature data;
preprocessing module 2: configured to preprocess the structured category feature data, structured numeric feature data and textual data for filling missing values in the structured category feature data and structured numeric feature data;
category vectorization module 3: configured to project the pre-processed structured category feature data in an Embedding module to obtain vectorized structured category feature data;
numerical vectorization module 4: configured to combine the pre-processed structured numerical feature data to obtain a combined matrix, and input the combined matrix into a preset linear layer for dimensionalization to obtain vectorized structured numerical feature data;
filtering module 5: configured to initialize a mask matrix with a structured numeric feature data based on the structured category feature data, take values on the initialized mask matrix with missing values padded in the structured numeric feature data according to the structured category feature data to obtain a target mask matrix;
weighted fusion module 6: for concatenating the vectorized structured category feature data and the vectorized structured numerical feature data to obtain structured features, inputting the structured features into a pre-built transformer model, performing feature construction on the structured features through a multi-head self-attention mechanism of the pre-built transformer model, calculating weight scores by filtering out the influence of missing features through the target mask matrix, performing weighted fusion of the projected structured features according to the weight scores, and obtaining output features of the pre-built transformer model;
structured data semantic acquisition module 7: configured to take the output features of the pre-built transformer model as input to the gated mlp module and the output features of the gated mlp module as global semantic features of structured data;
textual data semantic acquisition module 8: configured to input the textual data into a pre-trained language model, encode the textual data by the pre-trained language model, take features output by the pre-trained language model as semantic features of the textual data;
prediction module 9: configured to stitch global semantic features of the structured data and semantic features of textual data resulting in fused features, input the fused features into a linear layer through which probabilities of user medical rare events are output.

It will be appreciated that by the data acquisition module 1 for acquiring textual data and structured data relating to a patient from an electronic medical record, the structured data including structured category feature data and structured numerical feature data; by a preprocessing module 2 for preprocessing the structured category feature data, structured numeric feature data and textual data for filling missing values in the structured category feature data and structured numeric feature data; by the category vectorization module 3 for projecting the pre-processed structured category feature data in the Embedding module, resulting in vectorized structured category feature data; by the numerical vectorization module 4 for combining the pre-processed structured numerical feature data to obtain a combined matrix, inputting the combined matrix into a preset linear layer for dimensionalization to obtain vectorized structured numerical feature data; by a filtering module 5 for initializing a mask matrix based on the structured category feature data with structured numeric feature data, taking values on the initialized mask matrix according to the structured category feature data with missing values padded in the structured numeric feature data to obtain a target mask matrix; by a weighted fusion module 6 for concatenating the vectorized structured category feature data and the vectorized structured numerical feature data to obtain structured features, inputting the structured features into a pre-built transformer model, performing feature construction on the structured features through a multi-head self-attention mechanism of the pre-built transformer model, calculating weight scores by filtering out the influence of missing features through the target mask matrix, performing weighted fusion of the projected structured features according to the weight scores, and obtaining output features of the pre-built transformer model; by the structured data semantic acquisition module 7 configured to take the output features of the pre-built transformer model as input to the gated mlp module and the output features of the gated mlp module as global semantic features of the structured data; by a textual data semantic acquisition module 8 for inputting the textual data into a pre-trained language model, encoding the textual data by the pre-trained language model, taking features output by the pre-trained language model as semantic features of the textual data; the global semantic features of the structured data and the semantic features of the textual data are concatenating by a prediction module 9, resulting in fused features, which are input into a linear layer through which the probabilities of the medical rare events of the user are output.

### Embodiment Three

The embodiment provides a storage medium storing a computer program which, when executed by a master, implements each step in the above method.

It is understood that the above-mentioned storage medium may be a read-only memory, a magnetic disk or an optical disk, or the like.

It is to be understood that the same or similar parts of the above-described embodiments may be referred to with respect to each other, and that items not described in detail in some embodiments may refer to the same or similar items in other embodiments.

It should be noted that in the description of the present disclosure, the terms “first,” “second,” and the like are used for descriptive purposes only, and are not to be construed as indicating or implying relative importance, in the description of the present disclosure, unless otherwise specified, the meaning of “plurality” means at least two.

Any process or method descriptions in flow charts or otherwise described herein can be understood as, represents a module, token, or portion of code that includes one or more executable instructions for implementing specific logical functions or steps of a process, and the scope of the preferred embodiments of the present disclosure includes additional implementations in which functions may be executed out of order from that shown or discussed, including substantially concurrently or in reverse order depending on the functionality involved, as would be understood by those skilled in the art to which embodiments of the present disclosure pertain.

It is to be understood that portions of the present disclosure may be implemented in hardware, software, firmware, or a combination thereof. In the embodiments described above, various steps or methods may be implemented in software or firmware stored in memory and executed by a suitable instruction execution system. For example, if implemented in hardware, as in another embodiment, it can be implemented with any one or a combination of the following technologies which are well known in the art: a discrete logic circuit having logic gates for implementing logic functions on data signals, an application specific integrated circuit having suitable combinational logic gates, a programmable gate array (PGA), a field programmable gate array (FPGA), etc.

It will be appreciated by those skilled in the art that all or some of the steps carried by the methods to implement the embodiments described above may be performed by hardware associated with instructions from a program stored on a computer-readable storage medium that, when executed, includes one or a combination of the steps of the method embodiments.

Furthermore, each functional unit in each embodiment of the present disclosure may be integrated in one processing module, each unit may be physically present alone, or two or more units may be integrated in one module. The integrated modules described above may be implemented in the form of hardware or in the form of software functional modules. The integrated module may also be stored in one computer-readable storage medium if implemented in the form of a software functional module and sold or used as an independent product.

The above-mentioned storage medium may be a read-only memory, a magnetic disk or an optical disk, or the like.

In the description in this specification, reference to “one embodiment”, “some embodiments”, “an example”, “a specific example”, “some examples”, or the like means that a particular feature, structure, material, or characteristic described in connection with the embodiment or example is included in at least one embodiment or example of the present disclosure. In this specification, schematic representations of the aforementioned terms do not necessarily refer to the same embodiment or example. Furthermore, the particular features, structures, materials, or characteristics described may be combined in a suitable manner in any one or more embodiments or examples.

Although embodiments of the present disclosure have been shown and described above, it will be appreciated that the above embodiments are illustrative and not intended to be limiting of the present disclosure, and that one of ordinary skill in the art may make changes, modifications, substitutions, and variations to the above embodiments within the scope of the present disclosure.

1. A method for medical rare event prediction of multimodal data, comprising:
acquiring textual data and structured data related to patients from electronic medical records, the structured data comprises structured category feature data and structured numerical feature data;
pre-processing the structured category feature data, structured numeric feature data, and textual data for filling missing values in the structured category feature data and structured numeric feature data;
projecting the pre-processed structured category feature data in Embedding module to obtain vectorized structured category feature data;
combining the pre-processed structured numerical feature data to obtain a combined matrix, inputting the combined matrix into a preset linear layer for dimensionalization to obtain vectorized structured numerical feature data;
initializing a mask matrix with structured numerical feature data based on the structured category feature data, taking values of the initialized mask matrix with missing values imputed in structured numeric feature data according to the structured category feature data to obtain a target mask matrix;
concatenating the vectorized structured category feature data and the vectorized structured numerical feature data to obtain structured features, inputting the structured features into a pre-built transformer model, performing feature construction on the structured features through a multi-head self-attention mechanism of the pre-built transformer model, calculating weight scores by filtering out the influence of missing features through the target mask matrix, performing weighted fusion of the projected structured features according to the weight scores, and obtaining output features of the pre-built transformer model;
taking the output features of the pre-built transformer model as inputs to a gated mlp module and taking the output features of the gated mlp module as global semantic features of the structured data;
inputting the textual data into a pre-trained language model, encoding the textual data by the pre-trained language model, taking features output by the pre-trained language model as semantic features of the textual data;
concatenating global semantic features of the structured data and semantic features of textual data resulting in fused features, inputting the fused features into a linear layer through which probabilities of user medical rare events are output.

2. The method according to claim 1, wherein pre-processing the structured category feature data, the structured numeric feature data, and the textual data to fill missing values in the structured category feature data and the structured numeric feature data comprises:
converting character type of each category feature in the structured category feature data to an integer type, each category being represented by a different integer;
when a missing value occurs for any one category feature, populating with the category total for that category feature;
populating by the median of each numerical feature in the structured numerical feature data when missing values occur;
performing a mean-standard deviation normalization process on the populated structured numerical feature data;
extracting numerical features in the textual data by way of regular expression matching, adding the extracted numerical features to the structured numerical feature data.

3. The method according to claim 2, wherein projecting the pre-processed structured category feature data in an Embedding module resulting in vectorized structured category feature data comprises:
adding an offset to each class feature in the pre-processed structured class feature data, the offset being equal to a total number of classes of own pre-class features;
taking the values of a shifted class features as indices to retrieve corresponding row vectors in the Embedding matrix of the Embedding module, projecting all of the shifted class features to a matrix of a target dimension, and taking a matrix of the target dimension as vectorized structured class feature data.

4. The method according to claim 3, wherein feature construction of the structured features by a multi-head self-attention mechanism of the pre-built transformer model, calculating weight scores by filtering out the influence of missing features through the target mask matrix, performing weighted fusion of the projected structured features according to a weight scores, and obtaining an output features of the pre-built transformer model comprises:
the multi-head self-attention mechanism of the transformer model initializes three linear layers, the three linear layers comprising a parameter matrix Wq, a parameter matrix Wk, and a parameter matrix Wv, respectively, the three linear layers further comprising biases bq, bk, and bv, respectively;
projecting three linear layers of the structured features by their own parameter matrices and biases, resulting in a query vector, a key vector, and a value vector, respectively;
and performing a weighted fusion by the query vector, key vector, value vector, and weight scores to obtain the output features of the pre-built transformer model.

5. The method according to claim 4, wherein inputting the textual data into a pre-trained language model, encoding the textual data by the pre-trained language model, taking features output by the pre-trained language model as semantic features of the textual data comprises:
performing word tokenization on the textual data to obtain a word tokenization sequence, and adding special word tokenization [CLS] and [SEP] at the beginning and end of the word tokenization sequence, respectively;
converting each token into a corresponding token id according to a lexicon of the pre-trained language model, indexing a word vector of each token from an embedding matrix of the pre-trained language model with the token id;
inputting each word vector plus a corresponding position coding into the pre-trained language model, extracting features of textual data by a self-attention module, and taking word vectors F_text output by [CLS] word tokenization as semantic features of an entire piece of textual data.

6. The method according to claim 5, wherein initializing a mask matrix with structured numeric feature data based on the structured category feature data, taking the initializing mask matrix with missing values imputed in the structured numeric feature data according to the structured category feature data to obtain a target mask matrix comprises:
if n class features are included in the structured class feature data, m numerical features are included in the structured numerical feature data, the initialization mask matrix has a size of (n+m)×(n+m), each element in the initialized mask matrix takes a value of 0 or 1 on a condition that: M (i, j) represents whether a semantic relatedness between an i-th feature and a j-th feature needs to be calculated, when the j-th feature is missing, then let M (i, j)=0, otherwise M (i, j)=1, taking all elements of the initialized mask matrix after obtaining a target mask matrix.

7. An apparatus for medical rare event prediction of multimodal data, comprising:
a data acquisition module to acquire textual data and structured data related to a patient from electronic medical records, the structured data comprises structured category feature data and structured numerical feature data;
a preprocessing module for preprocessing the structured category feature data, structured numeric feature data and textual data for filling missing values in the structured category feature data and structured numeric feature data;
a category vectorization module: configured to project the pre-processed structured category feature data in the Embedding module, resulting in vectorized structured category feature data;
a numerical vectorization module: configured to combine the pre-processed structured numerical feature data to obtain a combined matrix, and input the combined matrix into a preset linear layer for dimensionalization to obtain vectorized structured numerical feature data;
a filtering module: configured to initialize a mask matrix with a structured numeric feature data based on the structured category feature data, take values on the initialized mask matrix with missing values padded in the structured numeric feature data according to the structured category feature data to obtain a target mask matrix;
a weighted fusion module: for concatenating the vectorized structured category feature data and the vectorized structured numerical feature data to obtain structured features, inputting the structured features into a pre-built transformer model, performing feature construction on the structured features through a multi-head self-attention mechanism of the pre-built transformer model, calculating weight scores by filtering out the influence of missing features through the target mask matrix, performing weighted fusion of the projected structured features according to the weight scores, and obtaining output features of the pre-built transformer model;
a structured data semantic acquisition module configured to take the output features of the pre-built transformer model as input to the gated mlp module and the output features of the gated mlp module as global semantic features of the structured data;
a textual data semantic acquisition module: configured to input the textual data into a pre-trained language model, encode the textual data by the pre-trained language model, take features output by the pre-trained language model as semantic features of the textual data;
a prediction module for concatenating global semantic features of the structured data and semantic features of textual data resulting in fused features, inputting the fused features into a linear layer through which probabilities of user medical rare events are output.

8. A storage medium, wherein storing a computer program, when executed by a master, implements various steps in a medical rare event prediction method of multimodal data according to claim 1.
